(19) United States
(12) Plant Patent Application Publication
(10) Pub. No.: US 2004/0064864 P1
(43) Pub. Date: Apr. 1, 2004

(54) Hydrangea macroophylla named 'Lady in Red'

(76) Inventor: Michael A. Dirr, Watkinsville, GA (US)

Correspondence Address: JINAN GLASGOW, P O BOX 28539, RALEIGH, NC, 276118539

(21) Appl. No.: 10/262,453

(22) Filed: Oct. 1, 2002

(51) Int. Cl. A01H 5/00

(52) U.S. Cl. PLT/250

(57) ABSTRACT

Hydrangea macrophylla ‘Lady in Red’ is a lacecap with the sterile florets of the inflorescences maturing from pale pink to burgundy rose. It has lustrous reddish-purple leaf venation, petiole and stem color, and the foliage develops a red-purple fall color. The plant has high mildew resistance.

### CROSS-REFERENCE TO RELATED APPLICATIONS

None applicable

### STATEMENT AS TO RIGHTS TO INVENTIONS MADE UNDER FEDERALLY-SPONSORED RESEARCH AND DEVELOPMENT

Not applicable

### BACKGROUND OF THE INVENTION

1. Field of the Invention

The present invention relates to a new and distinct variety of Hydrangea macrophylla (Thunb.) Ser., a member of the Hydrangeaceae family. Hydrangea macrophylla ‘LADY IN RED’ is the result of a controlled breeding program at the University of Georgia, Athens, Ga. and the Center for Applied Nursery Research, Dearing, Ga. The variety originated from open-pollinated H. m. ‘Otaksa’, and was selected from 265 seedlings for continued evaluation. The plant is used primarily as an ornamental. The variety, ‘LADY IN RED’, has been asexually reproduced by cuttings for 2 years. The novel leaf, stem, flower, and mildew resistance were consistent through these generations.

2. Description of Relevant Prior Art

‘LADY IN RED’ is distinguished from its parent (a mophead) and all other varieties of Hydrangea macrophylla by its lustrous reddish-purple stem, leaf venation, petiole color, reddish-purple fall color and high mildew resistance. Only Hydrangea macrophylla ‘Nigra’ (black-purple stems) and ‘Preziosa’ (reddish-purple stems) are noted for rich stem color. However, both have mophead flowers, are highly mildew susceptible, and do not develop the rich reddish-purple fall color.

### SUMMARY OF THE INVENTION

Hydrangea macrophylla ‘LADY IN RED’ develops lustrous reddish-purple stems, leaf venation and petiole coloration. The leaves turn reddish-purple in fall and are highly resistant to mildew. The lacecap sterile florets (sepals) open pinkish-white maturing to burgundy-rose.

### BRIEF DESCRIPTION OF DRAWINGS

The accompanying illustrations show characteristics of the new cultivar in photographs as true to color as is reasonably possible to make in illustrations of this nature.

FIG. 1 shows a 3-year-old unpruned plant in a 27 liter container

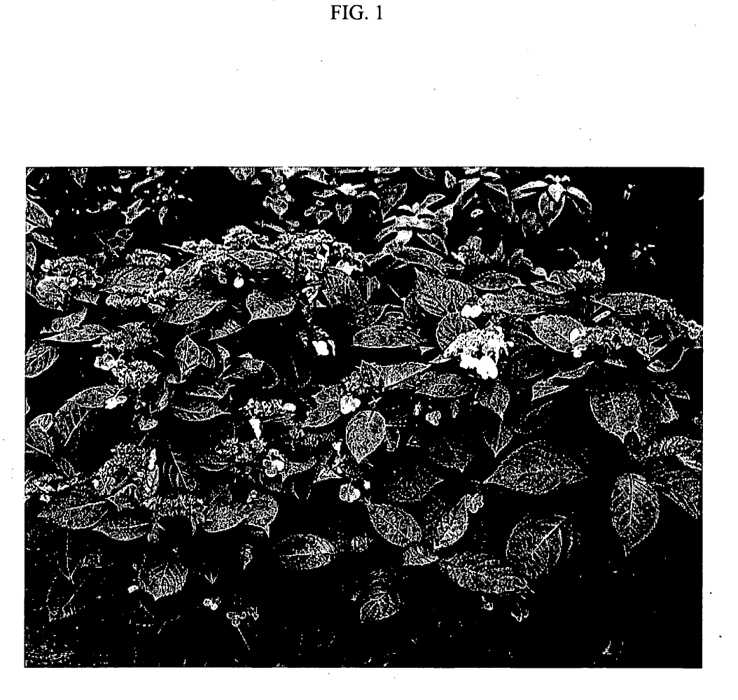

FIG. 2 illustrates the summer leaf color and texture

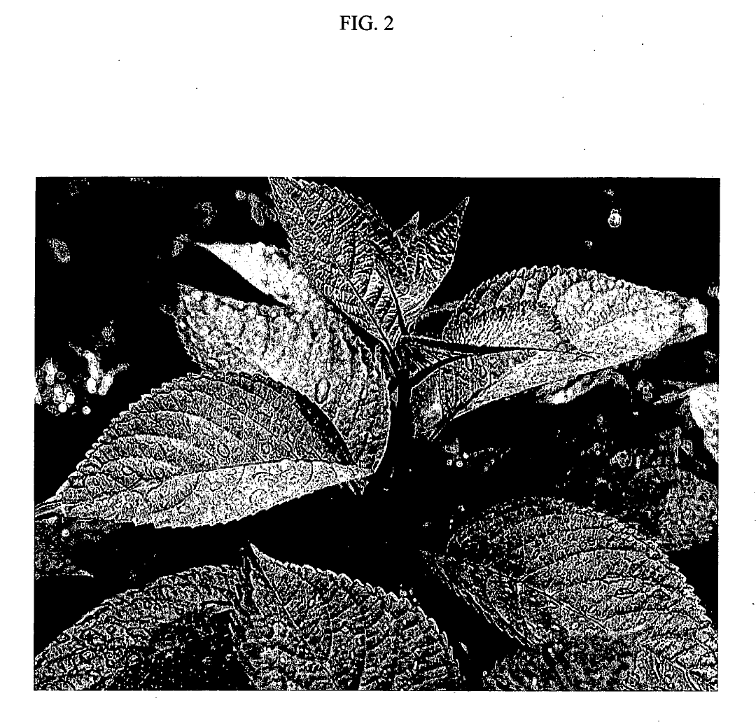

FIG. 3 illustrates the lower surface of the leaf with the prominent, reddish-purple raised veins

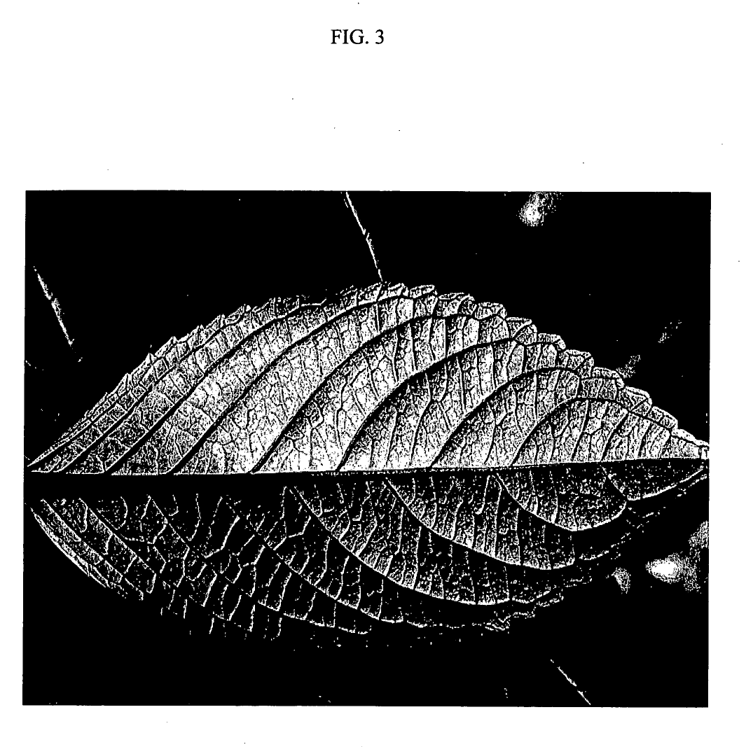

FIG. 4 illustrates the reddish-purple stem and petiole

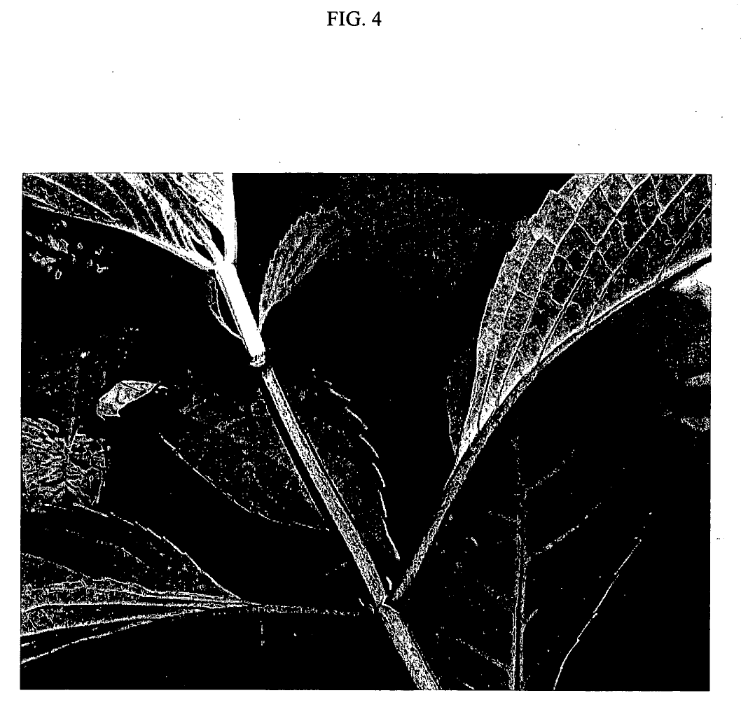

FIG. 5 illustrates the reddish-purple fall color

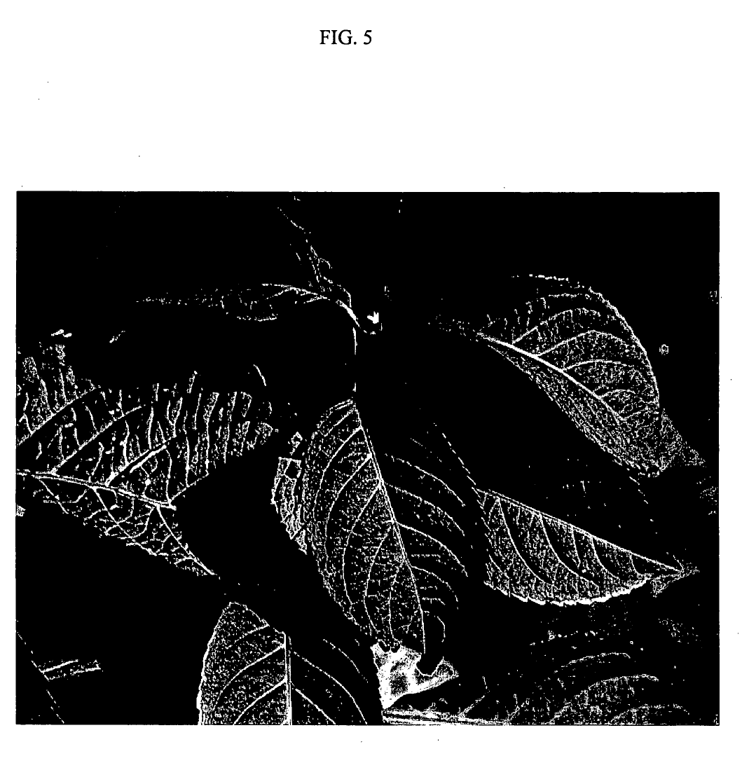

FIG. 6 illustrates the burgundy-rose mature sepals

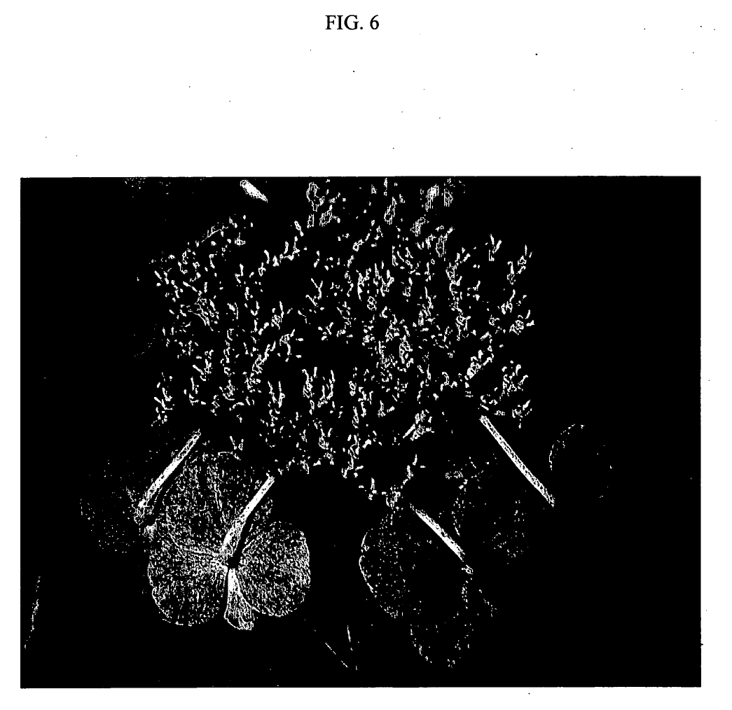

### BOTANICAL DESCRIPTION OF THE PLANT

A detailed description of Hydrangea macrophylla ‘LADY IN RED’ follows. Colors are based on The Royal Horticultural Color Chart (1995). All measurements/characteristics were taken from a 3-year-old plant in a 26.6 liter nursery container under 50% shade at Dearing, Ga. Measurements of leaves/stems and floral characteristics are based on 10 to 20 samples.

Classification:

Botanical.—Hydrangea macrophylla (Thunb.) Ser. ‘LADY IN RED’.

Parentage.—Open-pollinated seedling of Hydrangea macrophylla ‘Otaksa’.

Propagation.—Vegetatively by cuttings.

Plant:

Size.—58 cm high, 91.4 cm wide in 3-years.

Habit.—Mounded deciduous shrub, multistemmed and extremely compact.

Branching.—Many breaks (shoots) from base of one-year plants, ie, freely branching.

Leaf:

Shape.—Ovate.

Base.—Cuneate.

Apex.—Acute to abruptly acuminate.

Size.—Length 12.4 cm, width 6.7 cm.

Arrangement.—Opposite.

Margin.—Coarsely serrate.

Texture/substance.—Thickish; glabrous on upper and lower surfaces; eight prominent raised veins on lower surface.

Petioles.—2.4 cm long, glabrous, grooved above, rounded below. Color: Greyed-Purple Group 183A.

Mature leaf color.—Summer color varies with nutrition and intensity of sunlight. Upper leaf surface typically Yellow-Green Group 147A; lower surface Green Group 138B. Fall color is Red-Purple Group 59A. Petiole and lower leaf surface veins are Greyed-Purple Group 183A.

Stems:

Thickness.—0.5 cm to 0.6 cm diameter.

Texture.—Stout, terete, glabrous, lustrous.

Internodes.—5.2 cm.

Color.—Greyed-Purple Group 183A.

Hardiness.—USDA (1990) Zone 7 ( 17.8 C to 12.2 C) to 9 (6.7 C to 1.1 C).

Vigor.—Vigorous; rooted cuttings transplanted in spring develop a full 11.4 liter container by fall (Athens, Ga.).

Inflorescence:

Bloom period.—May to August, Athens and Dearing, Ga.

Flower arrangement.—Corymb, lacecap, 150 to 200 fertile flowers in center, 5 to 10 sterile florets around periphery.

Shape of inflorescence.—Flat-topped.

Inflorescence size.—10 to 15 cm wide.

Fertile flowers.—Glabrous, 5 ovate, entire sepals, acute at apex and base, each 0.4 cm long, 0.2 cm wide, entire flower 0.8 cm diameter, pedicel 0.6 cm long, flower buds globose, 0.4 cm high, 0.4 cm wide, color of buds and open petals Violet Group 84B.

Sterile florets.—3 to 4 sepals per cluster (showy floret), each rounded, entire, 1.6 cm long, 1.6 cm wide, collection of 3 to 4 sepals, 2.5 cm to 3.2 cm diameter.

Color.—Sterile florets: Upper surface — Purple Group 75C; lower surface — White Group 155D. Sepals, after fertile flowers are pollinated, turn Yellow-Green Group 144A on upper surface, Red Group 46A on lower surface.

Fragrance.—None noted.

Persistence of flowers.—Fertile flowers open over a 2 to 3 week period; sterile florets are effective for 2 to 3 months.

Reproductive system.—

Fertile flowers.—Five stamens on fertile flowers; pollen: white; stigma: 3 per ovary.

Fruit.—Capsule, urn-shaped, 0.5 cm long, 0.3 cm wide, Brown Group 200B.

I claim:

1. A new, distinctive, highly mildew resistant lacecap hydrangea, with lustrous reddish-purple leaf venation, petioles and stems, which develops burgundy rose florets and red-purple foliage fall color.
